(19) United States
(12) Patent Application Publication
(10) Pub. No.: US 2011/0071419 A1
(43) Pub. Date: Mar. 24, 2011

(54) LOCATION INDICATING DEVICE

(75) Inventors: Yan Liu, Eindhoven (NL); Bastiaan Wilhelmus Maria Moeskops, Eindhoven (NL); Alexander Franciscus Kolen, Eindhoven (NL); Femke Wagemakers, Eindhoven (NL); Golo Von Basum, Eindhoven (NL)

(73) Assignee: KONINKLIJKE PHILIPS ELECTRONICS N.V., EINDHOVEN (NL)

(21) Appl. No.: 12/992,635

(22) PCT Filed: May 20, 2009
(86) PCT No.: PCT/IB09/52093

§ 371 Date: Nov. 15, 2010

(30) Foreign Application Priority Data
May 26, 2008 (EP) 08156882.6

(51) Int. Cl. A61B 5/053

(52) U.S. Cl. 600/547

(57) ABSTRACT

The present invention provides a location indicating device (10) for determining on skin of a human or animal body a location for performing measurement of a physiological parameter in and/or treatment of the human or animal body. The location indicating device (10) comprises a matrix (7) of electrodes for determining skin resistance at different locations on skin of a human or animal body and a calculating unit (8) for calculating a location with highest skin resistance. The location indicating device (10) furthermore comprises visualisation means (9) for indicating the location of highest skin resistance to a user. The present invention also provides a method for making such a location indicating device (10) and a method for determining on skin of a human or animal body a location for performing measurement of a physiological parameter in and/or treatment of the human or animal body by using such a location indicating device (10).

### TECHNICAL FIELD OF THE INVENTION

The present invention relates to a location indicating device. More particularly, the present invention relates to a location indicating device for determining on skin of a human or animal body a location for performing measurement, e.g. invasive measurement of a physiological parameter in the human or animal body, to a method for making such a location indicating device and to a method for determining on skin of a human or animal body a location for performing measurement, e.g. invasive measurement of a physiological parameter in the human or animal body using such a location indicating device.

### BACKGROUND OF THE INVENTION

Measurement of physiological parameters of a body fluid of a human being or an animal, e.g. the presence and/or concentration of particular analytes in blood or interstitial fluids, are frequently performed by invasive measurement techniques, i.e. by measurement techniques which require piercing of skin of the human being or animal. For example, finger pricking may be used for invasive glucose monitoring or piercing skin on an arm of a human being may be done for blood sample taking. An undesirable effect of finger pricking or of invasive measurement techniques in general, is the pain or discomfort that it may cause and the corresponding inconvenience for the patient. In case of finger pricking this may be due to a high number of tactile nerve cells in the hand. The inconvenience is especially experienced in case the invasive measurements have to be repeated daily or even a few times each day. This may be the case for insulin-dependent people with diabetes who need to analyse their blood a few times a day. This inconvenient and painful procedure is a major reason why, in some cases, patients may skip some of the needed glucose analyses resulting in insufficient disease management.

Although different makeshift measures exist, such as e.g. the use of narcotics or local anesthesia, these measures do not make daily performance of invasive measurements more convenient for a patient as they may cause unwanted side effects.

### SUMMARY OF THE INVENTION

It is an object of embodiments of the present invention to provide a location indicating device for determining on skin of a human or animal body a location for performing measurement of a physiological parameter in the human or animal body, a method for making such a location indicating device and a method for determining on skin of a human or animal body a location for performing measurement of a physiological parameter in the human or animal body using such location indicating device.

The above objective is accomplished by a method and device according to the present invention.

A location indicating device and methods according to embodiments of the invention allows determining a location on skin of a human being or animal such that subsequent measurement of a physiological parameter, e.g. glucose concentration in blood which requires taking of a blood sample by piercing the skin with a needle, causes minimal pain or discomfort to the human being or animal.

The possibility to determine such physiological parameters, e.g. taking blood samples without or with minimum pain or discomfort may motivate people with diseases which require daily measurement of such physiological parameters, such as e.g. diabetes, for a better disease management. Moreover, the use of a device according to embodiments of the invention may remove some of the burdens parents may have when their kids suffer from such diseases. Hence, by using the location indicating device and methods according to embodiments of the invention discomfort of painful interventions to obtain information, e.g. information on parameter values in e.g. blood or interstitial fluids, from a human body or an animal body may be reduced. Furthermore, this pain and discomfort reduction may be achieved without the use of drugs and thus without uncomfortable and annoying side effects associated with such drugs.

A location indicating device and methods according to embodiments of the invention may be used with any known technique used for determining a physiological parameter for which piercing of the skin is required, e.g. piercing of the skin with a needle for taking a blood sample.

A location indicating device according to embodiments of the invention is easy to use. It can easily be used by the patient itself, and intervention of a doctor or nurse is not required.

In a first aspect, the present invention provides a location indicating device for determining on skin of a human or animal body a location for performing measurement of a physiological parameter in and/or treatment of the human or animal body, the location indicating device comprising:

a matrix of electrodes for determining skin resistance at different locations on skin of a human or animal body,

a calculating unit for determining a location with locally highest skin resistance, and

visualization means for indicating the location of locally highest skin resistance to a user.

A location indicating device according to embodiments of the invention allows determining a location on skin of a human being or animal such that subsequent measurement of a physiological parameter and/or treatment causes minimal pain or discomfort to the human being or animal. The possibility to determine such physiological parameters and to perform treatment, e.g. taking blood samples without or with minimum pain or discomfort may motivate people with diseases which require daily measurement of such physiological parameters, such as e.g. diabetes, for a better disease management. Moreover, the use of a device according to embodiments of the invention may remove some of the burdens parents may have when their kids suffer from such diseases. Hence, by using the location indicating device according to embodiments of the invention discomfort of painful interventions to obtain information, e.g. information on parameter values in e.g. blood or interstitial fluids, from a human body or an animal body may be reduced. Furthermore, this pain reduction may be achieved without the use of drugs and thus without uncomfortable and annoying side effects associated with these drugs.

According to embodiments of the invention, the matrix of electrodes may comprise a plurality of electrode pads and a counter electrode.

The calculating unit may, for example, comprise a microprocessor.

The visualization means may, for example, comprise a plurality of stamps for providing a dye onto the location with locally highest skin resistance or may comprise any other suitable visualisation means known by a person skilled in the art.

The present invention provides in a second aspect the use of a location indicating device according to embodiments of the invention for determining an appropriate location on a human or animal body for taking a blood sample.

In a further aspect, the present invention provides a method for making a location indicating device for determining on skin of a human or animal body a location for performing measurement of a physiological parameter in and/or treatment of the human or animal body, the method comprising:

providing a matrix of electrodes adapted for determining skin resistance at different locations on skin of a human or animal body,

providing a calculating unit adapted for determining a location with locally highest skin resistance, and

providing visualisation means for indicating the location of locally highest skin resistance to a user.

Providing visualisation means may, for example, be performed by providing a plurality of stamps for providing a dye onto the location with highest skin resistance.

The present invention also provides a location indicating device made by a method according to embodiments of the invention.

In still a further aspect, the present invention provides a method for determining on skin of a human or animal body a location for performing measurement of a physiological parameter in and/or treatment of the human or animal body, the method comprising:

determining skin resistance at different locations on skin of a human or animal body,

from the locations with determined skin resistance, determining a location with highest resistance, and

communicating this location with highest resistance to a user.

Determining skin resistance at different locations on skin of a human being or animal may comprise:

applying a voltage to electrode pads of a matrix of electrode pads, the matrix being applied to the skin,

measuring current flowing through a counter electrode as a consequence of the applied voltage, and

from the measured current determining skin resistance.

Communicating the location with highest resistance to a user may comprise providing a visual indication on the skin by means of a stamp comprising a dye.

The present invention furthermore provides the use of a method according to embodiments of the invention for determining a location on a human or animal body for taking a blood sample.

In yet a further aspect, the present invention provides a controller for controlled driving of electrodes of an electrode matrix of a location indicating device for determining on skin of a human or animal body a location for performing measurement of a physical parameter in and/or treatment of the human or animal body, the controller comprising a control unit for controlling a voltage source for driving at least one electrode of the electrode matrix.

The present invention also provides a computer program product for performing, when executed on a computing means, a method according to embodiments of the invention.

The present invention also provides a machine readable data storage device storing the computer program product according to embodiments of the invention.

The present invention also provides transmission of the computer program product according to embodiments of the invention over a local or wide area telecommunications network.

Particular and preferred aspects of the invention are set out in the accompanying independent and dependent claims. Features from the dependent claims may be combined with features of the independent claims and with features of other dependent claims as appropriate and not merely as explicitly set out in the claims.

The above and other characteristics, features and advantages of the present invention will become apparent from the following detailed description, taken in conjunction with the accompanying drawings, which illustrate, by way of example, the principles of the invention. This description is given for the sake of example only, without limiting the scope of the invention. The reference figures quoted below refer to the attached drawings.

### BRIEF DESCRIPTION OF THE DRAWINGS

FIG. 1 to FIG. 5 illustrate an experimental study to come to the insight of the present invention.

FIG. 6 illustrates a location indicating device according to an embodiment of the present invention.

FIG. 7 illustrates an electrode matrix that can be used with a location indicating device according to embodiments of the present invention.

FIG. 8 schematically illustrates a system controller for use with a location indicating device according to embodiments of the present invention.

FIG. 9 is a schematic representation of a processing system as can be used for performing a method according to embodiments of the present invention.

In the different figures, the same reference signs refer to the same or analogous elements.

### DESCRIPTION OF ILLUSTRATIVE EMBODIMENTS

The present invention will be described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes.

Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

In the description provided herein, numerous specific details are set forth. However, it is understood that embodiments of the invention may be practiced without these specific details. In other instances, well-known methods, structures and techniques have not been shown in detail in order not to obscure an understanding of this description.

The present invention provides a location indicating device for determining, on skin of a human or animal body, a location for performing measurement of a physiological parameter in and/or treatment of the human or animal body, a method for making such a location indicating device and a method for determining on skin of a human or animal body a location for performing measurement of a physiological parameter in and/or treatment of the human or animal body.

The physiological parameter may, for example, be any physiological parameter to be determined in a body fluid such as blood or an interstitial fluid (also referred to as tissue fluid or intercellular fluid). For example, the physiological parameter may be related to the presence and/or concentration of an analyte present in a body fluid such as blood or an interstitial fluid. The analyte may be any analyte of which it is important to detect its presence and/or to determine its concentration in the body fluid. An example hereof is the concentration of glucose in the blood of a human being. The analyte can be any organic molecule which is present in a human or animal body such as, for example, cholesterol, hemoglobin, acetone, water, lactic acid or melanin, or can be any inorganic molecule in a human or animal body such as, for example, iron or calcium, or can be another feature such as, for example the presence and/or concentration of gases or pH.

Embodiments of the present invention can be generally applied to any sensing method or treating method, or a combination of both. Examples of such sensing methods may, for example, be ultrasound, temperature sensing, pressure sensing, measurements using parts of the electromagnetic spectrum (such as optical, microwave or radiowave methods), skin impedance and capacitance measurements, and measurements of flux of compounds (such as TransEpidermal Water Loss). Examples of such treating methods may, for example, be any treating method to be applied to skin of a human or animal body, such as heat or light for hair removal, treatments for skin disorder or skin aging, any treating method to be applied through skin of a human or animal body, such as medication injection or transdermal drug delivery or any method to be applied into skin, such as using catheter ablation or taking biopsy.

According to other embodiments of the invention, the physiological parameter may also be a parameter suitable for determining skin properties such as e.g. skin cancer or skin aging. In such cases, the parameter may, for example, be reflectivity, evenness, temperature, temperature difference, color, color differences, stains.

As an example, parameters for determining skin properties such as skin cancer and skin aging could be optical properties of the skin. These may, among others, comprise performing measurements of absorption, scattering, reflection or birefringence at one or more wavelengths.

A location indicating device according to embodiments of the invention allows determining a location on skin of a human being or animal such that subsequent measurement of a physiological parameter, e.g. glucose concentration in blood which requires taking of a blood sample by piercing the skin with a needle, and/or treatment causes minimal pain or discomfort to the human being or animal.

The possibility to determine such physiological parameters and/or to perform treatment without or with minimum pain or discomfort may motivate people with diseases which require daily measurement of such physiological parameters and/or treatment, such as e.g. diabetes for a better disease management. Moreover, the use of a device according to embodiments of the invention may remove some of the burdens parents may have when their kids suffer from such diseases. Hence, by using the location indicating device and methods according to embodiments of the invention discomfort of painful interventions to obtain information, e.g. information on parameter values in, for example, blood or interstitial fluids, from a human body or an animal body may be reduced. Furthermore, this pain and discomfort reduction may be achieved without the use of drugs and thus without uncomfortable and annoying side effects associated with such drugs.

A location indicating device and methods according to embodiments of the invention may be used with any known invasive technique used for determining a physiological parameter for which piercing of the skin is required, e.g. piercing of the skin with a needle for taking a blood sample, and/or for treatment or the skin. In other words, the location indicating device and methods according to embodiments of the invention may be used with any known, invasive technique for determining a physiological parameter.

The location indicating device and methods according to embodiments of the invention are based on the use of impedance measurements to determine skin resistance at different locations. The use of such impedance measurements on skin of human beings or animals is known for different purposes by a person skilled in the art.

In “Newly explored electrical properties of normal skin and special skin sites”, (Biomedizinische Technik 49 (5), 117-124, May 2004) it is described that skin impedance measurements at various skin sites yield different impedance loci for normal skin and special skin sites. The results of skin impedance measurements taken at such sites with a two-electrode measurement system are presented in this article. Some of these sites can be identified as acupuncture points. Data from four volunteers were acquired by means of a data acquisition board and a measuring system consisting of the measurement circuit, including several electrode types, and a power supply. The results described in this article show that the measured parameters differ between normal skin and special skin sites. These effects have not previously been discovered or described, since there has been no systematic investigation of many acupuncture points to date, and there has been no apparent need for such an investigation. A number of necessary criteria for acupuncture point detection can be derived from the results obtained.

In “In vivo impedance measurements on nerves and surrounding skeletal muscles in rats and human body” (Medical & Biological Engineering & Computing 2002, Vol. 40), impedance measurements were used to find locations of nerves under human skin. In vivo impedance measurements were performed on exposed nervous and muscular tissues of rats. Similarly, such impedance measurements were also performed on the skin of six men, over the median nerve at the wrist, as well as 4 to 5 mm away from this location. Results obtained with rats have shown that the relative permittivity and conductivity of nerves are larger (by almost two orders of magnitude) than those observed for the muscular tissues surrounding the nerve. The results obtained on human skin in the frequency range of 20-200 kHz, when the electrodes were placed over the nerve, show lower resistance and higher capacitance than in the other areas measured. These preliminary results indicate that it may be possible to use impedance measurements to find the location of exposed nerves and also nerves under the skin.

According to embodiments of the present invention, impedance measurements are used for determining skin resistance of skin of a human being or an animal. Preliminary studies performed by the inventors revealed that a correlation exists between skin resistance at a particular location and pain or discomfort experienced by a human being or animal, also referred to as touch threshold, when the skin is pierced at that location. From these experiments it is assumed that locations with lowest skin resistance correspond to a high density of touch or pain receptors at those locations, and consequently to a high degree of pain or discomfort. Consequently, a location with highest skin resistance is assumed to be less sensitive to touch or pain. The location indicating device and methods according to embodiments of the invention rely on this insight that at locations on a skin of a human or animal body showing highest resistance, the least pain or discomfort is experienced by a human being or an animal when invasive measurements and/or treatments are performed to the body, for example when a needle is provided into the skin for e.g. taking a blood sample.

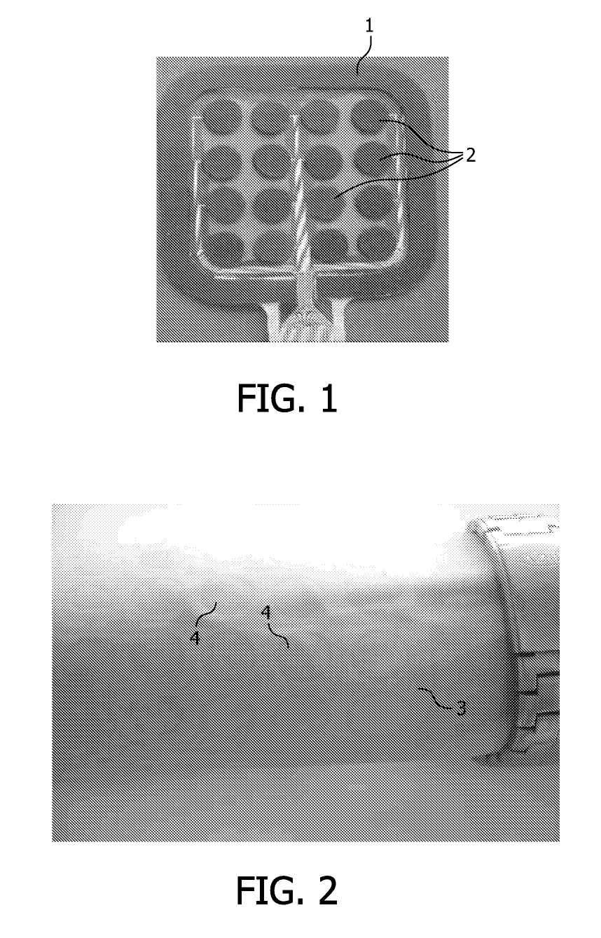

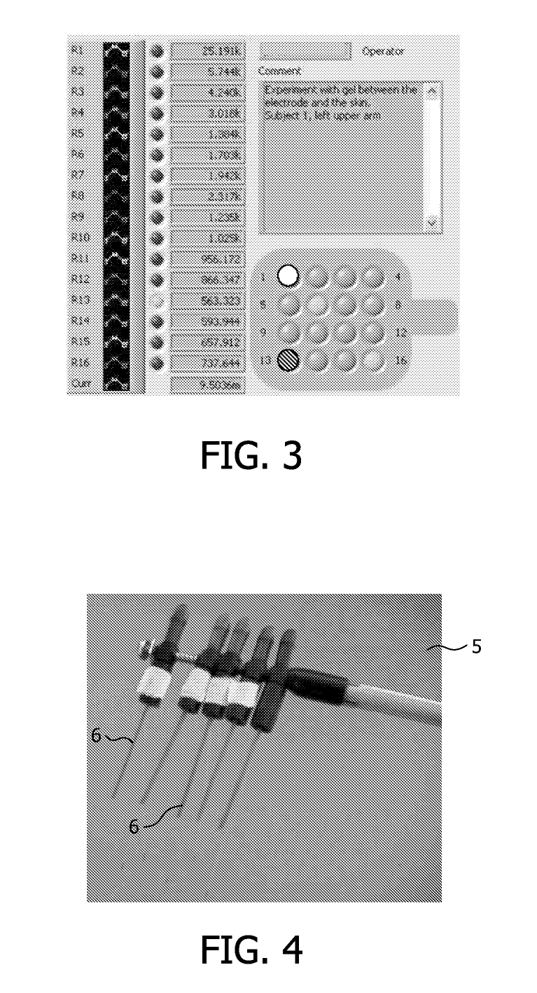

For demonstrating the above-described insight, a skin-resistance measuring electrode matrix from a transcutaneous electronic nerve stimulation (TENS) device was used. Such a TENS device is shown in FIG. 1. The TENS device comprises a reference electrode 1 surrounding a matrix of electrodes 2, also referred to as spot electrodes. For performing the study, measuring sites without any hair, e.g. at the volar part of a forearm 3 (see FIG. 2), were cleaned properly, and a little bit of gel was provided onto the skin at the measurement sites to enhance contact between the measuring sites on the forearm 3 and the electrodes 2 of the electrode matrix of the TENS device. By means of an elastic band (not shown) it was made sure that the electrode matrix tightly contacted the skin. Then, skin resistance measurements were performed. After the skin resistance measurements, the electrode matrix was heavily pressed onto the skin so that the skin was marked by the spot electrodes 2 as shown in FIG. 2. Some of the marked locations are indicated by reference number 4. Then two different locations were marked, i.e. respectively the location with lowest resistance and the location with highest resistance, on which then further tactile sensitivity measurements were performed (see further). Feedback on the feeling, i.e. on pain, was given by the person on whom these experiments were performed. The tests were conducted under single-blinded conditions at constant temperature and humidity. Results of the skin resistance measurements at different locations are shown in FIG. 3. The location with lowest resistance (in the example given No. 13, dashed circle) and the location with highest resistance (in the example given No. 1, white circle) were marked.

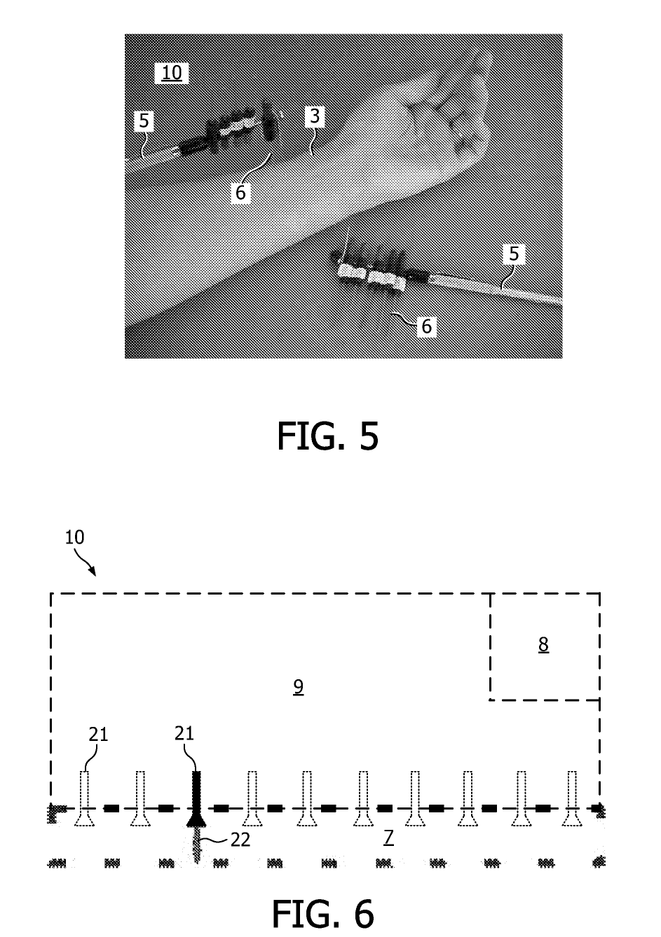

Next, an Esthesiometer was used for tactile sensitivity measurements to test or determine lower thresholds of touch or pain related with the locations where skin resistance was measured, more particularly related to the locations with respectively lowest and highest resistance. An example of such an Esthesiometer 5 is shown in FIG. 4. This tool 5 uses nylon mono filaments 6 with varying calibrated diameters, which are calibrated at gram/mm² pressure ratings, for applying different forces on the locations. The Esthesiometer was applied to the two specific locations, i.e. to the locations with highest and lowest resistance, to detect the lower threshold of touch or pain of each locations. Therefore, different forces were applied to the locations and the lower threshold of touch or pain was registered for the different locations. This is illustrated in FIG. 5. By doing so, the lowest touch or pain threshold was found at the locations with lowest resistance, i.e. at location No. 13, whereby the lower touch or pain threshold was detected at a force of only 0.150 gram/mm². Whereas at location No. 1, which was found to be the location with highest resistance, the test person could not feel any pain or discomfort until the force went up to 0.275 gram/mm² (see results in table 1.) A similar experiment as described above was performed again on the other arm of the same test person. A same correlation was again obtained between touch threshold and skin resistance. The results are:

TABLE 1
An example of threshold measurement results

| | | | Threshold |
|---|---|---|---|
| | Position No. | Resistance | (g/mm²) |
| Left volar | 13 | 52k | 0.15 |
| forearm | 1 | 200k | 0.275 |
| Right volar | 13 | 50k | 0.15 |
| forearm | 8 | >500k | 0.275 |

The results of the preliminary study have thus shown that a correlation exists between skin resistance and touch or pain threshold. They reveal that the lowest skin resistance most likely corresponds to the presence of a high density of touch/pain receptors and consequently to a low touch or pain threshold. On the other hand, the location with highest skin resistance proves to be less sensitive to touch/pain as it shows a higher touch or pain threshold than the location with lowest skin resistance.

The present invention uses this correlation between the skin resistance and lower pain threshold for determining a location on skin of a human or animal body for performing invasive measurements and/or treatments, e.g. for taking a blood sample by piercing a needle into the skin.

In a first aspect, the present invention provides a location indicating device for determining on skin of a human or animal body a location for performing measurement of a physiological parameter in and/or treatment of the human or animal body. The location indicating device comprises:

a matrix of electrodes for determining skin resistance at different locations on skin of a human or animal body,

a calculating unit for determining a location with highest skin resistance, and

visualisation means for indicating the location of highest skin resistance to a user.

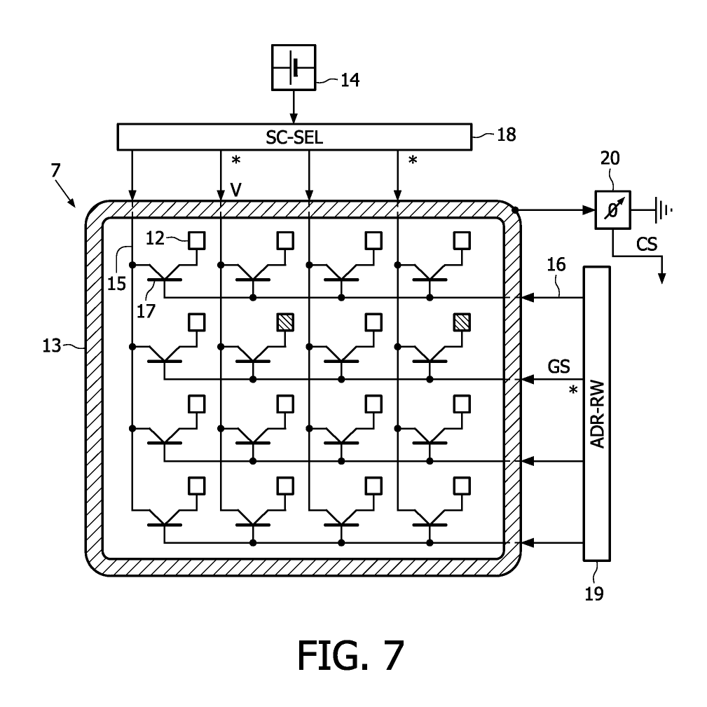

FIG. 6 schematically illustrates a location indicating device 10 according to an embodiment of the present invention. The location indicating device 10 comprises a matrix 7 of electrodes for determining skin resistance at different locations on skin of a human or animal body. The matrix 7 of electrodes may also be referred to as skin resistance mapping matrix. Skin resistance is determined by locally measuring electrical impedance. An example of an electrode matrix 7 that may be used with embodiments of the present invention is illustrated in FIG. 7. The electrode matrix 7 may comprise a plurality of electrodes pads 12 and a counter electrode 13 which is held at a reference voltage during measurement.

A voltage may locally be applied to the skin by activating a plurality of electrode pads 12, the number of electrode pads 12 being activated thereby depending on the size of the measurement region on the skin. Activation of the electrodes of the electrode matrix 7 may be such that, when it is placed on the skin, most or all of the activated electrode pads 12 and the counter electrode 13 make electrical contact with the skin. An activation voltage may be supplied by voltage source 14. For applying the voltage to the electrode pads 12, the electrode matrix 7 may furthermore comprise voltage lines 15 and addressing lines 16. Separate electrode pads 12 are coupled to one of the voltage lines 15 via respective switching elements such as e.g. thin-film transistors (TFTs) 17. Separate addressing lines 16 are coupled to the switching elements, e.g. TFTs 17, in respective rows of the matrix 7. The electrode matrix 7 may also comprise a source selection circuit 18 and a row addressing circuit 19. The source selection circuit 18 applies a voltage V to one or more selected voltage lines 15. The row addressing circuit 19 applies gate signals GS to one or more selected addressing lines 16. When a gate signal GS is applied to one of the addressing lines 16, the TFT 17 coupled to these addressing lines 16 is closed, i.e. rendered conductive. Hence, a voltage V is applied to electrode pads 12 which are coupled both to a voltage line 15 that is activated and to an addressing line 16 that is activated. For example, in FIG. 7 voltage lines 15 and addressing line 16 which are activated are indicated with an asterix (*). The electrode pads 12 which thereby are activated are indicated by dashed lines.

As a consequence of the applied voltage V to the electrode pads 12 an electrical current is received by the counter electrode 13. The electrical current is carried off to ground and is measured by a current meter 20, e.g. an ammeter. The current meter 20 generates a current signal CS whose signal level represents the measured current.

Further examples of electrode matrices 7 which can be used with embodiments of the present invention are described in WO 99/52588 A1.

The location indicating device 10 may furthermore comprise a calculating unit 8 (see FIG. 6). The current signal CS from the current meter 20 is then applied to the calculating unit 8. The calculating unit 8 then determines, e.g. by computing, the skin impedance at the respective electrode pads 12, derives local maximum values of the measured skin impedance and subsequently determines the electrode pad 12, and thus the location on the skin where this local maximum impedance value is measured. In other words, in that way the location with highest skin resistance is determined. According to the above-explained correlation between skin resistance and pain, this location then corresponds with the location which is less sensitive to pain when e.g. a needle is pierced through the skin at that location. Hence, this location may then be most suited for subsequently performing invasive measurements and/or treatments on.

The calculating unit 8 may include a computing device, e.g. microprocessor, for instance it may be a micro-controller. In particular, it may include a programmable computing device, for instance a programmable digital logic device such as a Programmable Array Logic (PAL), a Programmable Logic Array, a Programmable Gate Array, especially a Field Programmable Gate Array (FPGA). The use of an FPGA allows subsequent programming of the location indicating device, e.g. by downloading the required settings of the FPGA.

After determination of the location with highest resistance and thus least sensitive to touch or pain, this location has to be communicated to a user, e.g. a doctor, a patient and/or a nurse. This may be done by means of visualisation means 9. According to the example given in FIG. 6 the visualisation means 9 may comprise dye application means for applying a degradable dye to the location of the location with highest resistance. The dye application means may, for example, comprise a plurality of stamps 21 which can separately be driven so as to provide an amount of dye, e.g; degradable dye 22, onto the skin at the location with highest resistance (see FIG. 6). According to further embodiments of the invention, the visualisation means may comprise any means for providing the location of locally highest resistance with artificial markers which can be visualized by any optical means known by a person skilled in the art. For example, skin appearance parameters may be changed by, for example, stimulation. The visualisation means may use any methods using any feature of the measurement site, such as color, color changes, perfusion, perfusion changes, temperature by using e.g. Laser Doppler effects or an IR camera etc., to visualize the identified position, i.e. the location of locally highest resistance.

After the location with highest resistance, and thus least sensitive to pain, has been indicated, the location indicating device 10 may be removed from the skin. The location of highest resistance is then clear to the user, e.g. a doctor, a patient and/or a nurse, and subsequent measurement of a physiological parameter and/or treatment of the human or animal body may be performed. For example, a needle may be pierced through the skin at that location so as to, for example, take a blood sample.

By first using the location indicating device 10 according to embodiments of the invention, this subsequent measurement of the physiological parameter and/or treatment of the human or animal body may be performed with the patient experiencing a minimum of pain or discomfort. Furthermore, the location indicating device 10 according to embodiments of the invention is easy to use. It can easily be used by the patient itself, and intervention of a doctor or nurse is not required.

In a second aspect, the present invention provides a method for determining on skin of a human or animal body a location for performing measurement of a physiological parameter in and/or treatment of the human or animal body, the method comprising:

determining skin resistance at different locations on skin of a human or animal body,

determining a location with locally highest skin resistance, and

communicating this location with highest resistance to a user.

The method may be performed by using a location indicating device 10 as is described in the embodiments above. The location indicating device 10 comprises an electrode matrix 7, a calculating unit 8 and a visualisation means. The method will further be described by means of the electrode matrix 7 comprising a plurality of electrode pads 12 and a counter electrode 13. It has to be understood that this is not intended to limit the invention in any way and that any other suitable electrode matrix may be used with the method according to embodiments of the invention.

According to embodiments of the invention, measuring skin resistance at different locations on skin of a human being or animal may be performed as follows. First, the electrode pads 12 of the matrix 7 may be applied to the skin of the human being or animal. Next, a voltage may be applied to electrode pads 12 of the matrix 7. By applying a voltage to the electrode pads 12, a current will flow through the skin of the animal or human body to the counter electrode 13. The method may furthermore comprise measuring this current flowing through the counter electrode (13). In a next step, from the measured current skin resistance is then determined. In the skin impedance measurements, the skin resistance is the dominant term, especially the skin resistance at skin layers such as stratum corneum, epidermis and dermis. This is due to the skin structure, constitution and skin feature. The resistance of other terms, e.g. distance between electrodes, may be neglected compared to the skin resistance term, because a ratio between the resistance because of the distance between electrodes and the skin resistance may e.g. be 1/100.

Once skin resistance at different locations is measured and the location with locally highest resistance is determined, this is communicated to the user, e.g. the patient, doctor or nurse. Communicating the location with highest resistance to the user may comprise providing a visual indication on the skin, for example by means of a stamp 21 comprising a dye 22, e.g. degradable dye or by any other visualisation means as described above. In that way the location which is least sensitive to touch or pain is indicated and the user can easily and without too much pain or discomfort perform the invasive measurement and/or treatment of the human or animal body.

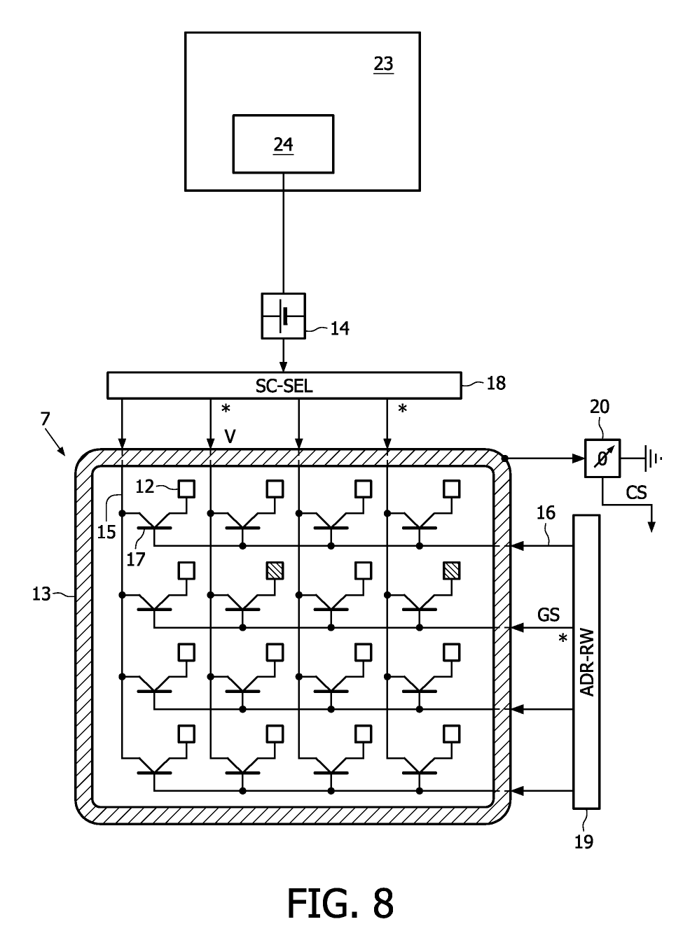

In a third aspect, the present invention also provides a system controller 23 for use in a location indicating device 10 according to embodiments of the present invention for controlled driving of electrodes of an electrode matrix 7 of the location indicating device 10. The system controller 23, which is schematically illustrated in FIG. 8, may comprise a control unit 24 for controlling a voltage source for driving at least one electrode of the electrode matrix 7, and for preferably subsequently driving different electrodes of the electrode matrix 7.

The system controller 23 may include a computing device, e.g. microprocessor, for instance it may be a micro-controller. In particular, it may include a programmable controller, for instance a programmable digital logic device such as a Programmable Array Logic (PAL), a Programmable Logic Array, a Programmable Gate Array, especially a Field Programmable Gate Array (FPGA). The use of an FPGA allows subsequent programming of the location indicating device 10, e.g. by downloading the required settings of the FPGA.

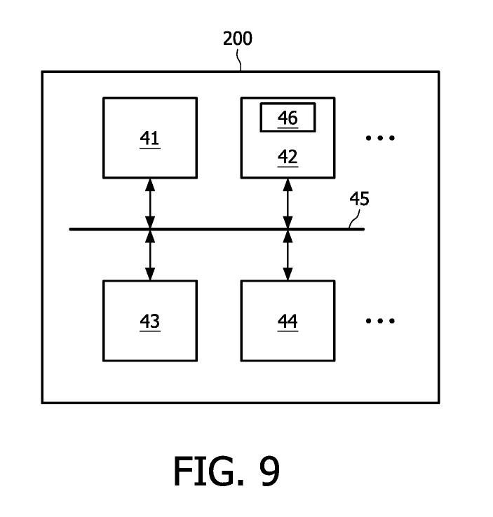

The method described above according to embodiments of the present invention may be implemented in a processing system 200 such as shown in FIG. 9. FIG. 9 shows one configuration of processing system 200 that includes at least one customizable or programmable processor 41 coupled to a memory subsystem 42 that includes at least one form of memory, e.g., RAM, ROM, and so forth. It is to be noted that the processor 41 or processors may be a general purpose, or a special purpose processor, and may be for inclusion in a device, e.g., a chip that has other components that perform other functions. Thus, one or more aspects of the method according to embodiments of the present invention can be implemented in digital electronic circuitry, or in computer hardware, firmware, software, or in combinations of them. The processing system may include a storage subsystem 43 that has at least one disk drive and/or CD-ROM drive and/or DVD drive. In some implementations, a display system, a keyboard, and a pointing device may be included as part of a user interface subsystem 44 to provide for a user to manually input information, such as parameter values. More elements such as network connections, interfaces to various devices, and so forth, may be included, but are not illustrated in FIG. 9. The various elements of the processing system 40 may be coupled in various ways, including via a bus subsystem 45 shown in FIG. 9 for simplicity as a single bus, but will be understood to those in the art to include a system of at least one bus. The memory of the memory subsystem 42 may at some time hold part or all (in either case shown as 46) of a set of instructions that when executed on the processing system 40 implement the steps of the method embodiments described herein.

The present invention also includes a computer program product which provides the functionality of any of the methods according to embodiments of the present invention when executed on a computing device. Such computer program product can be tangibly embodied in a carrier medium carrying machine-readable code for execution by a programmable processor. The present invention thus relates to a carrier medium carrying a computer program product that, when executed on computing means, provides instructions for executing any of the methods as described above. The term “carrier medium” refers to any medium that participates in providing instructions to a processor for execution. Such a medium may take many forms, including but not limited to, non-volatile media, and transmission media. Non-volatile media includes, for example, optical or magnetic disks, such as a storage device which is part of mass storage. Common forms of computer readable media include, a CD-ROM, a DVD, a flexible disk or floppy disk, a tape, a memory chip or cartridge or any other medium from which a computer can read. Various forms of computer readable media may be involved in carrying one or more sequences of one or more instructions to a processor for execution. The computer program product can also be transmitted via a carrier wave in a network, such as a LAN, a WAN or the Internet. Transmission media can take the form of acoustic or light waves, such as those generated during radio wave and infrared data communications. Transmission media include coaxial cables, copper wire and fibre optics, including the wires that comprise a bus within a computer.

It is to be understood that although preferred embodiments, specific constructions and configurations, as well as materials, have been discussed herein for devices according to the present invention, various changes or modifications in form and detail may be made without departing from the scope of this invention as defined by the appended claims.

1. Location indicating device for determining on skin of a human or animal body a location for performing measurement of a physiological parameter in and/or treatment on the human or animal body, the location indicating device comprising:
a matrix of electrodes for determining skin resistance at different locations on skin of a human or animal body,
a calculating unit for determining a location with locally highest skin resistance, and
visualisation means for indicating the location of locally highest skin resistance to a user.

2. Location indicating device according to claim 1, wherein the matrix of electrodes comprises a plurality of electrode pads and a counter electrode.

3. Location indicating device according to claim 1, wherein the calculating unit comprises a microcontroller.

4. Location indicating device according to claim 1 wherein the visualisation means comprises a plurality of stamps for providing a dye onto the location with locally highest skin resistance.

5. Use of a location indicating device according to claim 1 for determining an appropriate location on a human or animal body for taking a blood sample.

6. Method for making a location indicating device for determining on skin of a human or animal body a location for performing measurement of a physiological parameter in and/or treatment of the human or animal body, the method comprising:
providing a matrix of electrodes adapted for determining skin resistance at different locations on skin of a human or animal body,
providing a calculating unit adapted for determining a location with locally highest skin resistance, and
providing visualisation means for indicating the location of locally highest skin resistance to a user.

7. Method according to claim 6, wherein providing visualisation means is performed by providing a plurality of stamps for providing a dye onto the location with highest skin resistance.

8. Location indicating device made by a method according to claim 6.

9. Method for determining on skin of a human or animal body a location for performing measurement of a physiological parameter in and/or treatment of the human or animal body, the method comprising:
determining skin resistance at different locations on skin of a human or animal body,
from the locations with determined skin resistance, determining a location with highest resistance, and
communicating this location with highest resistance to a user.

10. Method according to claim 9, wherein determining skin resistance at different locations on skin of a human being or animal comprises:
applying a voltage to electrode pads of a matrix of electrode pads, the matrix being applied to the skin,
measuring current flowing through a counter electrode as a consequence of the applied voltage, and
from the measured current determining skin resistance.

11. Method according to claim 9, wherein communicating the location with highest resistance to a user comprises providing a visual indication on the skin by means of a stamp comprising a dye.

12. Use of a method according to claim 9 for determining a location on a human or animal body for taking a blood sample.

13. A controller for controlled driving of electrodes of an electrode matrix of a location indicating device for determining on skin of a human or animal body a location for performing measurement of a physical parameter in and/or treatment of the human or animal body, the controller comprising a control unit for controlling a voltage source for driving at least one electrode of the electrode matrix.

14. A computer program product for performing, when executed on a computing means, a method as in claim 9.

15. A machine readable data storage device storing the computer program product of claim 14.
